(19) United States
(12) Patent Application Publication
(10) Pub. No.: US 2022/0122742 A1
(43) Pub. Date: Apr. 21, 2022

(54) SYSTEM, DEVICE, METHOD, AND PROGRAM FOR ALLEVIATING SYMPTOM DEVELOPED IN ASSOCIATION WITH TREATMENT INVOLVING SIDE EFFECT OF DRUG

(71) Applicant: CureApp, Inc., Tokyio (JP)

(72) Inventors: Kohta Satake, Chuo-ku, Tokyo (JP); Masaki Aijima, Chuo-ku, Tokyo (JP)

(73) Assignee: CureApp, Inc., Tokyo (JP)

(21) Appl. No.: 17/423,670

(22) PCT Filed: Jan. 16, 2019
(86) PCT No.: PCT/JP2019/001085

§ 371 Date: Jul. 16, 2021

(51) Int. Cl. G16H 70/40; G16H 10/20; G16H 40/67; A61B 5/08

(52) U.S. Cl. CPC G16H 70/40; A61B 5/082; G16H 40/67; G16H 10/20

(57) ABSTRACT

There is provided a system for alleviating a symptom developed in association with a treatment involving a side effect of a drug.

The system is for alleviating the symptom developed in a user in association with the treatment involving the side effect of the drug. A server determines symptom scores for two or more respective symptoms based on user history information including information on a drug. The symptom scores indicate degrees of priority presented to the user. A user terminal presents information indicating the symptoms in descending order of the determined symptom scores to allow the user to select a symptom currently developed in the user, and receives a user selection indicating the symptom selected by the user based on the information indicating the presented symptoms. The server determines remedy information based on the user selection and the user history information. The remedy information indicates a remedy to alleviate the selected symptom. The user terminal presents the remedy to alleviate the symptom based on the determined remedy information.

### TECHNICAL FIELD

The present invention relates to a system, a device, a method, and a program for alleviating a symptom developed in association with a treatment involving a side effect of a drug.

### BACKGROUND ART

In the conventional medical procedure, a remedy for a symptom developed in association with a treatment involving a side effect of a drug was known only in a prior explanation and a consultation at the time of a medical examination by a healthcare provider. As development of information communication technology in recent years, using an information terminal, such as a smartphone, possessed by a prescribed person, the prescribed person can examine side effects and remedies of a therapeutic agent prescribed himself/herself using the information terminal by himself/herself. For example, Prior Literature 1 discloses a system for presenting side effect diseases assumed from a medication history as a risk close examination table. The use of this system allows identification that the side effect disease comes from which prescribed medical agent.

### CITATION LIST

### Patent Literature

Patent Document 1: JP 2018-147463 A

### SUMMARY OF INVENTION

### Technical Problem

While the information terminal treats a large volume of information, a size of a screen presenting the information to a user is limited. It is difficult for the prescribed person to instantaneously obtain the required information among various contexts in the screen having the limited size. In particular, it is extremely difficult to obtain information on the remedy using the information terminal in a case where a judgment ability decreases due to an effect of a symptom developed in association with a side effect or another treatment, or, for example, an input operation cannot be sufficiently performed due to, for example, numbness. That is, the prescribed person has a technical difficulty to easily acquire the symptoms and the remedies using the information terminal.

The system described in Prior Literature 1 does not provide a solution to determine which symptom should be preferentially presented to the prescribed person according to the prescribed person and consequently obtain an effective remedy even in a restricted environment.

### Solution to Problem

The present invention has been made in view of the problems described above, and has characteristics such as the following. That is, a system according to one embodiment of the present invention is a system that includes a server and a user terminal. The system is for alleviating a symptom developed in a user in association with a treatment involving a side effect of a drug. The server determines symptom scores for two or more respective symptoms based on user history information including information on a drug. The symptom scores indicate degrees of priority presented to the user. The user terminal presents information indicating the symptoms in descending order of the determined symptom scores to allow the user to select a symptom currently developed in the user, and receives a user selection indicating the symptom selected by the user based on the information indicating the presented symptoms. The server determines remedy information based on the user selection and the user history information. The remedy information indicates a remedy to alleviate the selected symptom. The user terminal presents the remedy to alleviate the symptom based on the determined remedy information.

The determining the symptom scores can include determining the symptom scores for the two or more respective symptoms based on state information. The state information indicates states of the user determined based on the user history information.

The server may store a score table. The score table may indicate the symptom scores for the respective symptoms predetermined for the respective states of the user. The determining the symptom scores may include selecting the symptom scores for the respective symptoms of the user from the score table based on the state information.

The server can change the predetermined symptom score for each of the symptoms based on the user selection.

The determining the remedy information may include determining remedy scores indicating levels of expected effects for two or more respective remedies based on state information indicating states of the user determined based on user history information, and selecting the remedy information indicating a remedy with a highest remedy score.

The server can store a score table. The score table can indicate the remedy scores for the respective remedies for the respective symptoms predetermined for respective states of the user. The determining the remedy information can include selecting the remedy with the highest remedy score of the user from the score table based on the user selection and the state information.

The server may change the predetermined remedy score for each of the remedies for each of the symptoms based on effect confirmation information. The effect confirmation information may indicate an effect of a remedy taken by the user.

The determining the symptom scores may include determining the symptom scores for the respective two or more symptoms based on presence or absence of behaviors of the user determined based on the user history information.

The determining the remedy information may include determining remedy scores that indicates levels of predicted effects for the two or more respective remedies based on presence or absence of behaviors of the user determined based on the user history information, and selecting the remedy information indicating a remedy with a highest remedy score.

The user history information may include at least one of attribute information, past medical history information, prescription information, biometric information, and behavior history information. The behavior history information may indicate behaviors practiced by the user.

The biometric information may include an exhaled air carbon monoxide concentration of the user measured by a carbon monoxide concentration measuring apparatus.

The remedy information may be further determined based on prescription information indicating a type and an amount of a drug prescribed to the user.

A server according to one aspect of the present invention is for alleviating a symptom developed in a user in association with a treatment involving a side effect of a drug. The server: determines symptom scores for two or more respective symptoms based on user history information including information on a drug, the symptom scores indicating degrees of priority presented to the user; transmits the symptom scores to a user terminal to present information indicating the symptoms in descending order of the symptom scores; receives a user selection indicating the symptom selected by the user based on the information indicating the presented symptoms from the user terminal; determines remedy information based on the user selection and the user history information, the remedy information indicating a remedy to alleviate the selected symptom; and transmits the remedy information to the user terminal.

A user terminal according to one aspect of the present invention is for alleviating a symptom developed in a user in association with a treatment involving a side effect of a drug. The user terminal: receives symptom scores for two or more respective symptoms determined based on user history information including information on a drug from a server, the symptom scores indicating degrees of priority presented to the user; presents information indicating the symptoms in descending order of the determined symptom scores to allow the user to select a symptom currently developed in the user; receives a user selection indicating the symptom selected by the user based on the information indicating the presented symptoms, transmits the user selection to the server to determine remedy information based on the user selection and the user history information, the remedy information indicating a remedy to alleviate the selected symptom; receives the determined remedy information from the server; and presents the remedy to alleviate the symptom based on the remedy information.

Further, a method according to one aspect of the present invention is for alleviating a symptom developed in a user in association with a treatment involving a side effect of a drug. The method is executed by one or more computers. The method includes: determining symptom scores for two or more respective symptoms based on user history information including information on a drug, the symptom scores indicating degrees of priority presented to the user; presenting information indicating side effects in descending order of the determined symptom scores to allow the user to select a symptom currently developed in the user; receiving a user selection indicating the side effect selected by the user based on the information indicating the presented symptoms; determining remedy information based on the user selection and the user history information, the remedy information indicating a remedy to alleviate the selected symptom; and presenting the remedy to alleviate the symptom based on the determined remedy information.

Further, a method according to one aspect of the present invention is for alleviating a symptom developed in a user in association with a treatment involving a side effect of a drug. The method is executed by a server. The method includes: determining symptom scores for two or more respective symptoms based on user history information including information on a drug, the symptom scores indicating degrees of priority presented to the user; transmitting the symptom scores to a user terminal to present information indicating the symptoms in descending order of the symptom scores; receiving a user selection indicating the symptom selected by the user based on the information indicating the presented symptoms from the user terminal; determining remedy information based on the user selection and the user history information, the remedy information indicating a remedy to alleviate the selected symptom; and transmitting the remedy information to the user terminal.

A method according to one aspect of the present invention is for alleviating a symptom developed in a user in association with a treatment involving a side effect of a drug. The method is executed by a user terminal. The method includes: receiving symptom scores for two or more respective symptoms determined based on user history information including information on a drug from a server, the symptom scores indicating degrees of priority presented to the user; presenting information indicating the symptoms in descending order of the determined symptom scores to allow the user to select a symptom currently developed in the user; receiving a user selection indicating the symptom selected by the user based on the information indicating the presented symptoms; transmitting the user selection to the server to determine remedy information based on the user selection and the user history information, the remedy information indicating a remedy to alleviate the selected symptom; receiving the determined remedy information from the server; and presenting the remedy to alleviate the symptom based on the remedy information.

As one aspect of the present invention, a set of programs that cause one or more computers to execute the above-described method can be configured.

### Advantageous Effects of Invention

The use of the present invention allows the user to easily select the symptom developed in association with a treatment and to perform the remedy believed to be effective to alleviate the selected symptom.

### BRIEF DESCRIPTION OF DRAWINGS

FIG. 1 is a configuration diagram of a system according to an embodiment of the present invention.

FIG. 2 is a hardware configuration diagram of a user terminal and a server according to an embodiment of the present invention.

FIG. 3 is a functional block diagram of the user terminal and the server according to an embodiment of the present invention.

FIG. 4A is an example of a correlation diagram between a user history and states according to an embodiment of the present invention.

FIG. 4B is an example of a correlation diagram between user histories and a state according to an embodiment of the present invention.

FIG. 5 is a flowchart according to an embodiment of the present invention.

FIG. 6 is an example of a user terminal screen according to an embodiment of the present invention.

FIG. 7 is an example of the user terminal screen according to an embodiment of the present invention.

FIG. 8 is an example of the user terminal screen according to an embodiment of the present invention.

FIG. 9 is an example of the user terminal according to an embodiment of the present invention.

### DESCRIPTION OF EMBODIMENTS

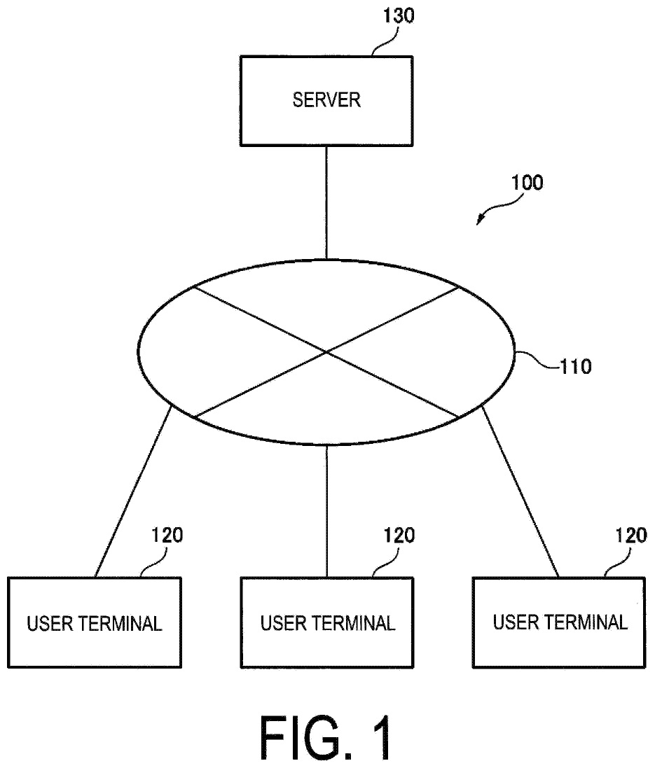

FIG. 1 illustrates an example of a system configuration diagram of the present invention. A system 100 is used for alleviating a symptom occurred through a treatment, and includes a network 110 and a user terminal 120 and a server 130 connected to the network 110.

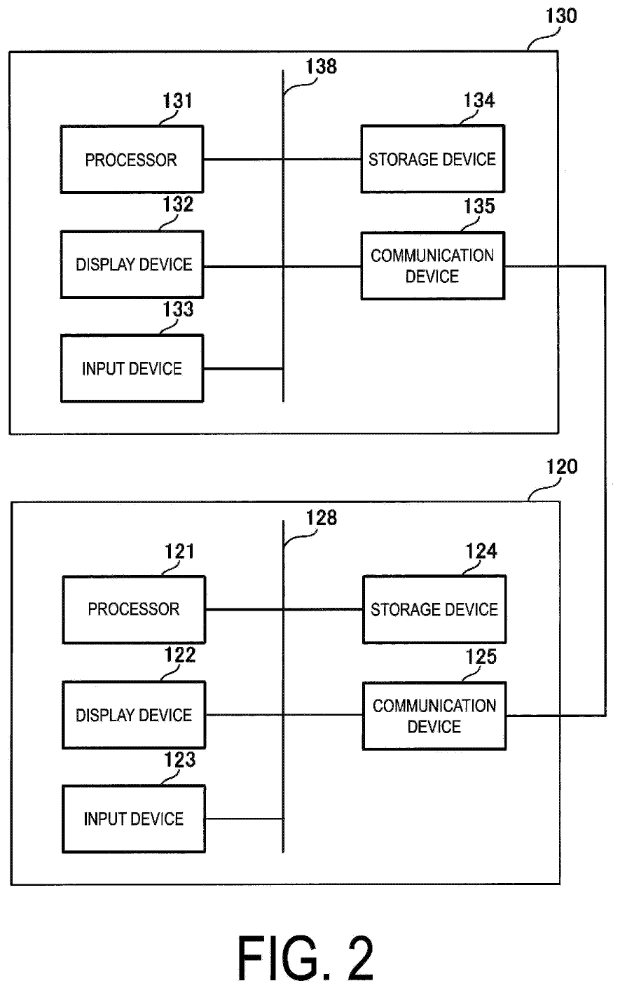

FIG. 2 is a block diagram illustrating a hardware configuration of the user terminal 120 and the server 130 according to an embodiment of the present invention. The user terminal 120 includes a processor 121, a display device 122, an input device 123, a storage device 124, and a communication device 125. The respective configuration devices are connected with a bus 128. It is assumed that an interface is interposed between the bus 128 and each configuration device as necessary. In the present embodiment, the user terminal 120 is a smartphone. However, as long as the above-described configuration is provided, the user terminal 120 can be an information terminal, such as a personal computer, a tablet computer, and a touchpad.

Similarly, the server 130 includes a processor 131, a display device 132, an input device 133, a storage device 134, and a communication device 135. The respective configuration devices are connected with a bus 138. It is assumed that an interface is interposed between the bus 138 and each configuration device as necessary. In the present embodiment, the server 130 is achieved by a computer.

The processors 121, 131 control the entire operation of the user terminal 120 and the entire server 130, and are, for example, CPUs. The processors 121, 131 read and execute programs and data stored in the storage devices 124, 134 to execute various processes. As one example, the processors 121, 131 are constituted of a plurality of processors.

In accordance with the control by the processors 121, 131, the display devices 122, 132 display, for example, application screens, to a user (a patient) of the user terminal 120 and a user (an administrator) of the server 130. The display devices 122, 132 are preferably liquid crystal displays, but may be, for example, displays using organic EL and plasma displays.

The input devices 123, 133 are user interfaces that receive inputs from the user to the user terminal 120 and the server 130, and are, for example, touch panels, touch pads, keyboards, or computer mice. Because the user terminal 120 is the smartphone in the present embodiment, the user terminal 120 includes a touch panel as the input device 123, the touch panel also functions as the display device 122, and the display device 122 and the input device 123 are integrated. The display device 122 and the input device 123 may be in a separate configuration disposed at different locations. Because the server 130 is the computer, it is assumed that the server 130 includes a keyboard and a computer mouse as input devices, and includes a liquid crystal display as a display device.

The storage devices 124, 134 are storage devices provided with a general smartphone and a computer including a RAM as a volatile memory and a ROM as a non-volatile memory. The storage devices 124, 134 can also include external memories. For example, the storage device 124 stores a user application for embodying the present embodiment, and the storage device 134 stores a server application. The user application includes user programs for alleviation of symptoms and various kinds of data referred to during the user program execution. The user program is started in response to an operation of the user on the user terminal 120, and is executed on an operating system (OS) implemented in the user terminal 120 in advance. A server application includes functions and various kinds of data such that reception of the input from the user and presentation of information to the user are properly performed on each user terminal 120 as a client by the user programs.

In one example, the storage devices 124, 134 include main storage devices and auxiliary storage devices. The main storage devices are volatile storage media capable of reading and writing information at high speed, and are used as storage areas and work areas when the processors 121, 131 process the information. The auxiliary storage devices store various programs and data used by the processors 121, 131 in execution of the respective programs. Although the auxiliary storage device is, for example, a hard disk device, as long as the auxiliary storage device can store the information, any non-volatile storage or a non-volatile memory may be used, and the auxiliary storage device may be attachable/removable. The auxiliary storage device stores, for example, an operating system (OS), middleware, an application program, and various kinds of data that are possibly referred to in association with the execution of these programs.

The communication devices 125, 135 transfer data with the other devices over the network 110 (omitted in FIG. 2). For example, the communication devices 125, 135 perform wireless communications, such as mobile communications or wireless LAN, and connect to the network 110. The user terminal 120 communicates with the server 130 via a network by the use of the communication device 125. The communication devices 125, 135 may perform wired communication using, for example, an Ethernet (registered trademark) cable.

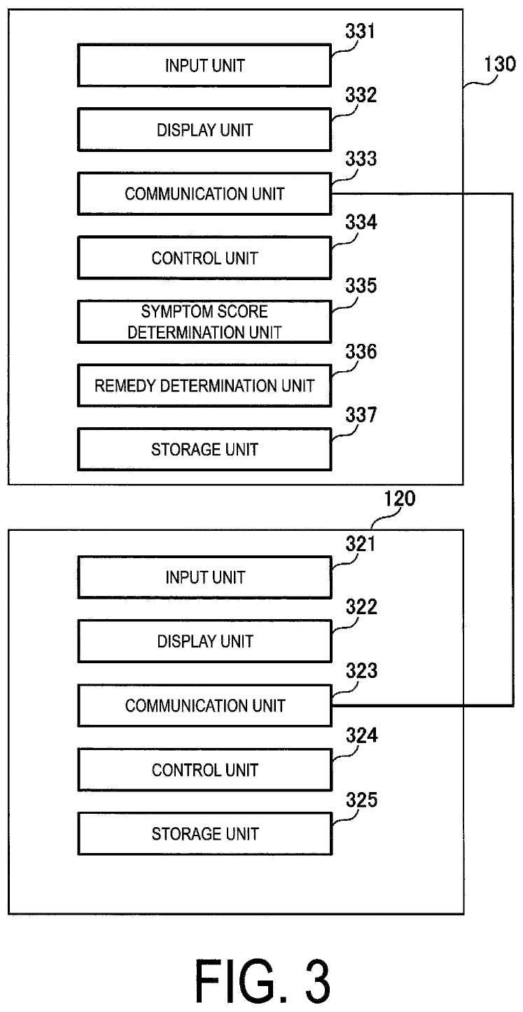

FIG. 3 is an example of a functional block diagram of the user terminal 120 and the server 130 of the present invention. The user terminal 120 includes an input unit 321, a display unit 322, a communication unit 323, a control unit 324, and a storage unit 325. The server 130 includes an input unit 331, a display unit 332, a communication unit 333, a control unit 334, a symptom score determination unit 335, a remedy determination unit 336, and a storage unit 337. The input units 321, 331 have a function of receiving inputs from the user. The display units 322, 332 have a function of displaying information so that the user can view the information. The communication units 323, 333 have a function of transmitting and receiving information to and from the other devices. The control units 324, 334 have a function of executing control, such as information processing. The storage units 325, 337 have a function of storing, for example, tables and data. The control units 324, 334 store data in the storage units 325, 337 and update the data in the storage units 325, 337. The symptom score determination unit 335 has a function of determining a score indicating a level of a degree of priority for presenting each symptom to the user. The remedy determination unit 336 has a function of determining a remedy believed to be highly effective to the user. In the present embodiment, these functions are achieved by the programs stored being executed in the storage devices 124, 134 in the processing devices 121, 131 illustrated in FIG. 1, and respective hardware and software operating in cooperation, but can be achieved by configuring, for example, an electronic circuit for achieving each function.

In the present embodiment, the description will be given with a case in which the user is a patient who takes a therapeutic agent for a smoking cessation treatment under a guidance by a healthcare provider as an example, but the present embodiment may be applied to a case of any disease or a case in which a plurality of diseases are simultaneously treated. In addition to a medical disease, such as a renal dysfunction, high blood pressure, and a mental illness, as long as the disease means a physically undesirable state, the disease need not be a disease in a medical sense. The treatment includes preventive medicine. The patient refers to a person who attempts to alleviate a symptom developed in association with a treatment using the present invention, and does not necessarily need to be a patient who gets a treatment for a disease under a guidance of a healthcare provider. The symptom developed in association with the treatment may be any symptom as long as it is a symptom developed in association with a treatment, such as a side effect of a drug and a poor health condition brought by a withdrawal symptom and a suffered symptom.

While the storage unit 337 in the server stores user history information regarding a history of the user in the present embodiment, the storage unit 325 in the user terminal 120 may store some or all pieces of the user history information. The user history information regarding the history of the user is history information related to the treatment of the user that can be used for inference of the symptom developed in the user in association with the treatment, and includes information indicating a history of the user other than behaviors and information indicating a behavior history. The information indicating the history of the user other than behaviors includes information related to drugs. The information related to the drugs includes prescription drug data. The prescription drug data is not necessarily limited to drugs prescribed by the healthcare provider, but includes information on drugs purchased in, for example, pharmacies by selection of the user himself/herself. The information indicating the history of the user other than behaviors can further include attribute data and past medical history data of the user (the patient). The data include, for example, information shown in Tables 1 to 3, are input using a personal computer (not illustrated) used by a doctor when the user visits the doctor for outpatient care as the healthcare provider to start a smoking cessation treatment, and can be stored in the storage unit 337 in the server.

TABLE 1
User Attribute Data

| User ID | Name | | Date of birth | Age | Start date |
|---|---|---|---|---|---|
| 1 | Yamada | Taro | 7/3/1978 | 40 | 10/1/2018 |
| 2 | Tanaka | Hanako | 1/3/1987 | 30 | 1/11/2017 |
| . . . | . . . | . . . | . . . | . . . | . . . |

As shown in Table 1, the user attribute data includes the name, the date of birth, the age, and the treatment start date of the user. The information is associated with the user ID and stored. The age may be updated at a predetermined timing based on the date of birth. The start date is the day when the treatment was started, for example, can be a day when the user visited for outpatient care for the first time.

TABLE 2
Past Medical History Data

| Data | Disease | | User | | |
|---|---|---|---|---|---|
| ID | ID | Disease name | ID | Activation | Timestamp |
| 1 | 1 | Interstitial | 1 | 1 | 2/21/2018 |
| | | pneumonia | | | |
| 2 | 2 | Chronic | 2 | 0 | 5/7/2017 |
| | | constipation | | | |
| 3 | 3 | High stress | 1 | 1 | 2/12/2018 |
| 4 | 4 | Sleep apnea | 2 | 1 | 2/15/2018 |
| | | syndrome | | | |
| 5 | 5 | Renal | 2 | 1 | 2/1/2018 |
| | | dysfunction | | | |
| 6 | 6 | Nicotine | 1 | 1 | 2/1/2018 |
| | | addiction | | | |
| . . . | . . . | . . . | . . . | . . . | . . . |

The past medical history data of the patient includes the past medical history data ID, the disease ID, the disease name, the user ID, the activation, and the timestamp. The information is associated with the user ID and stored. The disease ID is an ID assigned to each disease. Although it is omitted here, a disease table is separately prepared in advance and the disease ID is associated with the disease name and stored. For example, the doctor refers to the disease table and selects and inputs the disease that the patient suffers from, thus storing the disease as the past medical history data in the storage unit 337. “Activation” indicates whether the patient still currently suffers from the disease (active) or was healed (inactive). “1” means that the patient still currently suffers, and “0” means that the patient was healed. The timestamp can be a date when the information was input or a date when the information was updated. Unless otherwise stated, data IDs and timestamps of other data are similar.

TABLE 3
Prescription Drug Data

| Data | Drug | | User | | |
|---|---|---|---|---|---|
| ID | ID | Drug name | ID | Amount | Timestamp |
| 1 | 1 | Drug name A | 1 | 12 | 10/1/2018 |
| | | (Antiemetic) | | | |
| 2 | 2 | Drug name B | 1 | 24 | 10/1/2018 |
| | | (Smoking-cessation | | | |
| | | aid 1) | | | |
| 3 | 3 | Drug name C | 2 | 15 | 2/14/2018 |
| | | (Antidepressant) | | | |
| 4 | 4 | Drug name D | 3 | 5 | 2/14/2018 |
| | | (Smoking-cessation | | | |
| | | aid 2) | | | |
| . . . | . . . | . . . | . . . | . . . | . . . |

The prescription drug data includes the data ID, the drug ID, the prescription drug name, the user ID, the amount, and the timestamp. The drug ID is an ID assigned to each drug. Although it is omitted here, a drug table is separately prepared in advance and the drug ID is associated with the drug name and stored. For example, the doctor refers to the drug table and selects and inputs the drug prescribed to the patient, thus storing the drug as the prescription drug data in the storage unit. Although the explanations of the drugs are described in the parentheses following the drug names for understanding in the specification, they need not to be included in the data. The amount of drug indicates the amount of prescribed drug.

The storage unit 337 in the server further stores a behavior table. The behavior table is a list of behaviors possibly practiced by the user as the patient and defines the behaviors and the types of behavior. For example, the information shown in Table 4 can be included.

TABLE 4
Behavior Table

| Behavior | | |
|---|---|---|
| ID | Behavior | Type |
| 1 | Guided to stop taking the drug | Action |
| 2 | Guided to drink water | Action |
| 3 | Guided to cool down | Action |
| 4 | Guided to take the drug name A (antiemetic) | Action |
| 5 | Smoke cigarettes | Behavior to |
| | | be treated |
| 6 | Sleep | Basic behavior |
| 7 | Wake up | Basic behavior |
| 8 | Dose the drug name D (smoking-cessation | Taking a drug |
| | aid 2) | |
| 9 | Use the drug name B (smoking-cessation aid | Taking a drug |
| | 1) | |
| 10 | Dose the drug name A (antiemetic) | Taking a drug |
| 11 | Sleepy | Symptom |
| 12 | Got swollen | Symptom |
| 13 | Have nightmares | Symptom |
| 14 | Cannot sleep | Symptom |
| 15 | Want to smoke | Symptom |
| 16 | Feel nausea | Symptom |
| 17 | Have a headache | Symptom |
| 18 | Guided to consult with a doctor | Action |
| 19 | Feel irritated | Symptom |
| 20 | Dose the drug name C (antidepressant) | Taking a drug |
| . . . | . . . | . . . |

Behavior data includes the behavior ID, the behavior, and the type. “Behavior” indicates the content of behavior that the user possibly practices, “Type” indicates the type of the behavior, and the information is associated with the behavior ID and stored. In the present embodiment, the type of behavior includes the basic behavior, the behavior to be treated, taking a drug, the symptom, and the action. The basic behavior is the basic behavior that a person practices, like the behavior ID=6 “Sleep” or the behavior ID=7 “Wake up”. “Behavior to be treated” is the behavior of the user target for treatment. In the present embodiment, since the user undergoes a smoking cessation treatment, the behavior to be treated is “Smoke cigarettes”. “Taking a drug” is a behavior of dosing a drug. Here, the behavior IDs=8, 9, 10, and 20 are assigned to the behaviors of taking the drug name D (the smoking cessation agent 2), the drug name B (the smoking cessation agent 1), the drug name A (the antiemetic), and the drug name C (the antidepressant), respectively. “Symptom” means the symptom that develops in association with the treatment and includes the side effect of the drug. For example, the symptom “Feel nausea” is defined as the behavior ID=16. “Action” means a behavior to be practiced by the user as a remedy when a symptom occurs. For example, in a case where it is determined that the user should stop taking the drug for the symptom developed in the user, the behavior ID=1 is designated and the user is caused to take the remedy “Guided to stop taking the drug”. Thus, the user is expected to practice the behavior as the remedy, which is the stop of taking the drug.

Information indicating the behavior history of the user includes biometric information data and behavior history data. Table 5 and Table 6 show data as an example of them used in the present embodiment.

TABLE 5
Biometric Information Data

| Data | User | | | |
|---|---|---|---|---|
| ID | ID | Biometric information | Scale | Timestamp |
| 1 | 1 | Exhaled air carbon | 0.0 | 12/11/2018 12:12:01 |
| | | monoxide concentration | | |
| 2 | 1 | Exhaled air carbon | 15.0 | 12/12/2018 12:12:01 |
| | | monoxide concentration | | |
| 3 | 1 | Exhaled air carbon | 15.0 | 12/14/2018 12:12:00 |
| | | monoxide concentration | | |

TABLE 6
Behavior History Data

| Data | User | Behavior | | |
|---|---|---|---|---|
| ID | ID | ID | Behavior | Timestamp |
| 1 | 1 | 5 | Smoke cigarettes | 12/12/2018 12:12:01 |
| 2 | 1 | 9 | Apply the drug name B | 12/12/2018 23:12:01 |
| | | | (smoking-cessation aid 1) | |
| 3 | 1 | 9 | Apply the drug name B | 12/13/2018 11:12:01 |
| | | | (smoking-cessation aid 1) | |
| 4 | 1 | 5 | Smoke cigarettes | 12/14/2018 12:12:00 |
| 5 | 1 | 9 | Apply the drug name B | 12/14/2018 23:12:01 |
| | | | (smoking-cessation aid 1) | |
| 6 | 1 | 16 | Feel nausea | 12/14/2018 23:26:01 |

The biometric information data is data indicating biometric information of the user. As shown in Table 5, the biometric information data includes the data ID, the user ID, the biometric information, the scale, and the timestamp. “Biologic information” indicates the type of the biological information, and is a CO concentration contained in exhaled air of the patient measured by a carbon monoxide (CO) concentration measuring apparatus in the present embodiment, but may include other information, such as a blood glucose value or a blood pressure. The scale indicates the measured value, for example, indicates CO concentration=15.0 ppm in the data ID=2.

The behavior history data is data indicating the history of behaviors practiced by the user in which the user ID, the behavior ID, the behavior, and the timestamp are associated with the data ID and stored. The user terminal or the server can determine the behavior estimated as being practiced by the user based on the biometric information data and add it to the behavior history data. For example, in a case where the carbon monoxide (CO) concentration measuring apparatus measured the CO concentration contained in the exhaled air of the user and the CO concentration greater than or equal to a predetermined threshold (e.g., 8.0 ppm) was measured, the behavior ID=5 and the behavior=“Smoke cigarettes” can be added to the behavior history data. In this case, the time at which the CO concentration was measured can be the timestamp in the behavior history.

A state list table is a list of states possibly taken by the user and can include, for example, the information shown in Table 7.

TABLE 7
State List Table

| State | | Duration |
|---|---|---|
| ID | State | (h) |
| 1 | Smoking | 72 |
| 2 | Taking the drug name D (smoking-cessation aid | 24 |
| | 2) | |
| 3 | Using the drug name B (smoking-cessation aid | 24 |
| | 1) | |
| 4 | Taking the drug name A (antiemetic) | 24 |
| 5 | Keeping the drug name D (smoking-cessation | 288 |
| | aid 2) | |
| 6 | Keeping the drug name B (smoking-cessation | 288 |
| | aid 1) | |
| 7 | Keeping the drug name A (antiemetic) | 288 |
| 8 | Lack of sleep | 168 |
| 9 | Swollen | 24 |
| 10 | Having nightmares | 24 |
| 11 | Want to smoke | 24 |
| 12 | Feeling nausea | 24 |
| 13 | Having a headache | 24 |
| 14 | Affected with interstitial pneumonia | 0 |
| 15 | Suffer from chronic constipation | 0 |
| 16 | Under a high stress | 0 |
| 17 | Have a disorder in the kidney function | 0 |
| 18 | Addicted to nicotine | 0 |
| 19 | Feeling irritated | 0 |
| 20 | Taking the drug name C (antidepressant) | 24 |
| 21 | Keeping the drug name C (antidepressant) | 288 |
| . . . | . . . | . . . |

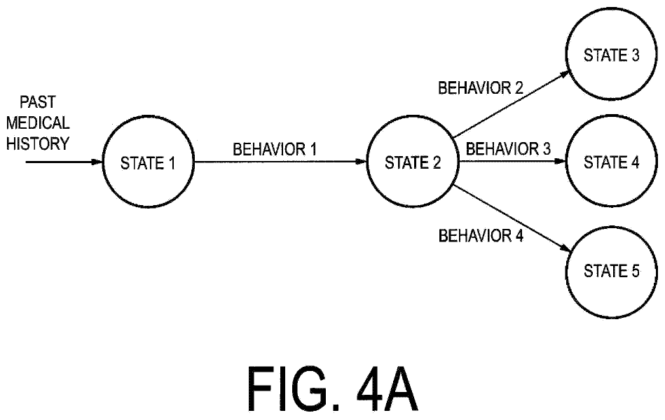

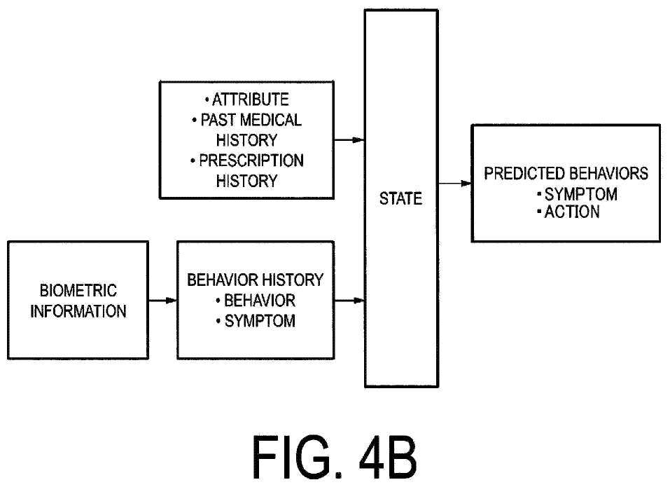

In Table 7, “State” indicates a state possibly taken by the user, and “Duration” is a duration during which the state is possibly maintained after the user is in the state. “0” means that the duration is not used. That is, it is not determined as inactive due to the elapsed time. As illustrated in FIG. 4, the state transitions by the history other than the behaviors, such as the attribute, the past medical history, or the prescription history, or the behavior history. For example, as illustrated in FIG. 4A, the user who entered “State 1” by a certain past medical history transitions to “State 2” by practicing “Behavior 1”. Further, the state of the user can transition to different states, “State 3” to “State 5”, by the behavior practiced next in “State 2”. As shown in FIG. 4B, the behavior history includes the behaviors practiced by the user and the symptoms developed in the user. Additionally, the behaviors practiced by the user can be determined based on the biometric information. The state of the user can be determined based on the user history including the attributes, the past medical history, the prescription history, and the behavior history, and the behavior practiced by the user next can be predicted based on the state. “Predicted behavior” is not limited to the behavior practiced by the intention of the user, and includes the symptoms developed in association with the treatment, for example, “Feel irritated” and “Feel nausea”. Determining the state of the user allows narrowing down the behaviors practiced by the user next and predicting the behavior.

In the present embodiment, the storage unit in the server further stores a history-to-state table. The history-to-state table is a table for determining the current state of the user based on the user history information. Table 8 shows an example of the history-to-state table.

TABLE 8
History-to-State Table

| State | | | |
|---|---|---|---|
| ID | State | History ID | History |
| 1 | Smoking | Behavior ID = 5 | Smoke cigarettes |
| 2 | Taking the drug name | Behavior ID = 8 | Dose the drug name |
| | D (smoking-cessation | | D (smoking-cessation |
| | aid 2) | | aid 2) |
| 3 | Using the drug name B | Behavior ID = 9 | Apply the drug name |
| | (smoking-cessation aid | | B (smoking-cessation |
| | 1) | | aid 1) |
| 4 | Taking the drug name | Behavior ID = 10 | Dose the drug name |
| | A (antiemetic) | | A (antiemetic) |
| 5 | Keeping the drug | Drug ID = 4 | Drug name D |
| | name D (smoking- | | (smoking-cessation aid |
| | cessation aid 2) | | 2) |
| 6 | Keeping the drug | Drug ID = 2 | Drug name B |
| | name B (smoking- | | (smoking-cessation aid |
| | cessation aid 1) | | 1) |
| 7 | Keeping the drug | Drug ID = 1 | Drug name A |
| | name A (antiemetic) | | (antiemetic) |
| 8 | Lack of sleep | Behavior ID = 11 | Sleepy |
| | | Behavior ID = 14 | Cannot Sleep |
| 9 | Swollen | Behavior ID = 12 | Got swollen |
| 10 | Having nightmares | Behavior ID = 13 | Have nightmares |
| 11 | Want to smoke | Behavior ID = 15 | Want to smoke |
| 12 | Feeling nausea | Behavior ID = 16 | Feel nausea |
| 13 | Having a headache | Behavior ID = 17 | Have a headache |
| 14 | Affected with | Disease ID = 1 | Interstitial pneumonia |
| | interstitial pneumonia | | |
| 15 | Suffer from chronic | Disease ID = 2 | Chronic constipation |
| | constipation | | |
| 16 | Under a high stress | Disease ID = 3 | High stress |
| 17 | Have a disorder in | Disease ID = 5 | Renal dysfunction |
| | the kidney function | | |
| 18 | Addicted to nicotine | Disease ID = 6 | Nicotine addiction |
| 19 | Feeling irritated | Behavior ID = 19 | Feel irritated |
| 20 | Taking the drug name | Behavior ID = 20 | Dose the drug name |
| | C (antidepressant) | | C (antidepressant) |
| 21 | Keeping the drug | Drug ID = 3 | Drug name C |
| | name C (anti- | | (antidepressant) |
| | depressant) | | |
| . . . | . . . | . . . | . . . |

The history-to-state table in the present embodiment includes the state ID, the state, the history ID, and the history. “State ID” is an ID of the state and “State” indicates the content of the state. “History ID” is an ID for identifying the history of the treatments of the user by the past medical history data, the prescription drug data, and the ID included in the behavior history data, and is any of the disease ID, the drug ID, and the behavior ID. “History” is the content of the history identified by the “History ID”. By associating the history ID with the state ID, in a case where the history identified by the history ID is present, it indicates that the user enters the state identified by the state ID. A plurality of the history IDs can be made correspond to one state ID. In the present embodiment, it is determined that the user enters the state when the user practices, for example, the behavior identified by one history ID among the plurality of corresponded history IDs. However, it can be determined that the user enters the state when the user practices, for example, all behaviors identified by the history IDs.

For example, it is found that by practicing the behavior of “Smoke cigarettes” as the behavior ID=5, the user enters the state “Smoking” as the state ID=1. Additionally, “Keeping the drug name B (smoking-cessation aid 1)” as the state ID=6 is associated with the “Drug name B (smoking cessation agent 1)” as the drug ID=2. This indicates that when the drug ID=2 in the prescription drug data, that is, the prescription of the drug name B (smoking cessation agent 1) is shown, the user keeps the drug name B (the smoking cessation agent 1).

In the present embodiment, the storage unit in the server further stores a behavior-to-state score table. The behavior-to-state score table defines a score given to each behavior based on the state of the user. Table 9 shows the behavior-to-state score table used in the present embodiment.

TABLE 9-1
Behavior-to-State Score Table

| Data | Behavior | | First state | | Second state | | Third state | |
|---|---|---|---|---|---|---|---|---|
| ID | ID | Score | ID | Score | ID | Score | ID | Score |
| 1 | 16 Feel | 0.8 | 3 Using | 1 | 1 Smoking | 1 | 16 Under | 1 |
| | nausea | | the drug name | | | | a high | |
| | | | B (smoking- | | | | stress | |
| | | | cessation aid 1) | | | | | |
| 2 | 16 | 0.4 | 3 | 1 | 1 | 0 | 16 | 1 |
| 3 | 16 | 0.4 | 3 | 0 | 1 | 1 | 16 | 1 |
| 4 | 16 | 0.3 | 3 | 0 | 1 | 0 | 16 | 1 |
| 5 | 16 | 0.7 | 3 | 1 | 1 | 1 | 16 | 0 |
| 6 | 16 | 0.3 | 3 | 1 | 1 | 0 | 16 | 0 |
| 7 | 16 | 0.3 | 3 | 0 | 1 | 1 | 16 | 0 |
| 8 | 16 | 0.2 | 3 | 0 | 1 | 0 | 16 | 0 |
| 9 | 17 Have | 0.6 | 3 | 1 | 1 | 1 | — | — |
| | a headache | | | | | | | |
| 10 | 17 | 0.4 | 3 | 1 | 1 | 0 | — | — |
| 11 | 17 | 0.4 | 3 | 0 | 1 | 1 | — | — |
| 12 | 17 | 0.3 | 3 | 0 | 1 | 0 | — | — |
| 13 | 4 Guided to | 0.8 | 12 Feeling | 1 | 7 Keeping | 1 | 4 Taking the | 0 |
| | take the | | nausea | | the drug | | drug name A | |
| | drug name A | | | | name A | | (antiemetic) | |
| | (antiemetic) | | | | (antiemetic) | | | |
| 14 | 4 | 0.0 | 12 | 1 | 7 | 1 | 4 | 1 |
| 15 | 4 | 0.0 | 12 | 1 | 7 | 0 | 4 | 0 |
| 16 | 4 | 0.0 | 12 | 0 | 7 | 1 | 4 | 1 |
| 17 | 1 Guided to | 0.7 | 12 | 1 | 7 | 1 | 4 | 1 |
| | stop taking | | | | | | | |
| | the drug | | | | | | | |
| 18 | 1 | 0.1 | 12 | 1 | 7 | 0 | 4 | 0 |
| 19 | 1 | 0.9 | 12 | 1 | 16 | 1 | — | — |
| 20 | 1 | 0.6 | 12 | 1 | 16 | 0 | — | — |
| 21 | 12 Got | 0.4 | 3 Using | 1 | — | — | — | — |
| | swollen | | the drug name | | | | | |
| | | | B (smoking- | | | | | |
| | | | cessation aid 1) | | | | | |

TABLE 9-2

| | | | | | | | | |
|---|---|---|---|---|---|---|---|---|
| 22 | 15 Want | 0.7 | 1 Smoking | 1 | — | — | — | — |
| | to smoke | | | | | | | |
| 23 | 5 Smoke | 0.8 | 16 Under | 1 | — | — | — | — |
| | cigarettes | | a high stress | | | | | |
| 24 | 19 Feel | 0.2 | 18 Addicted | 1 | 3 Using | 1 | 3 Smoking | 1 |
| | irritated | | to nicotine | | the drug name | | | |
| | | | | | B (smoking- | | | |
| | | | | | cessation aid 1) | | | |
| 25 | 19 Feel | 0.9 | 18 Addicted | 1 | 3 | 0 | 3 Smoking | 0 |
| | irritated | | to nicotine | | | | | |
| 26 | 18 Guided to | 0.9 | 12 | 1 | 17 Having | 1 | — | — |
| | consult with | | | | a renal | | | |
| | a doctor | | | | dysfunction | | | |
| 27 | 18 | 0.6 | 12 | 1 | 17 | 0 | — | — |
| . . . | . . . | . . . | . . . | . . . | . . . | . . . | . . . | . . . |

In Table 9, the behavior and the states are associated with the data ID, and the behavior and the respective first to third states include the IDs and the scores. The states associated with one behavior need not be the same. Behaviors and states corresponding to the IDs are described in some fields of the IDs in Table 9 for explanation. For example, the behavior ID of the data ID=1 is 16. According to the behavior table (Table 4), the behavior corresponding to this is “Feeling nausea” and therefore it is written in the field of the behavior ID for explanation. The scores in the first to the third state indicate whether the user is in the state corresponding to each state ID. The score=1 means that the user is in the state and the score=0 means that the user is not in the state. In the present embodiment, the score is 0 or 1, but 0 to 9 or other values can be given. For example, values from 0 to 9 indicating the level of stress can be given to the state “Under a high stress” in the state ID=16. There is no need to always assign the three states for each behavior. For example, although the three states are associated with the data IDs=1 to 8, only the two states are associated with the data ID=9.

The score for each behavior indicates a level of a degree of priority of the behavior when the state is in the corresponding first to the third states. When the behavior is a “Symptom”, the degree of priority of the symptom presented to the user is indicated. In a case where a possibility of causing the symptom is high, the degree of priority is set high. Since the symptom that is more likely to occur in the user is preferentially presented, the user can easily select the symptom that occurs himself/herself. Moreover, when there is a possibility that a symptom that limits an ability of the user to select the symptom, such as numbness or dizziness, or a symptom having a high risk of hazardous to life occurs, a high score may be given to these symptoms. Even when the ability of the user to select the symptom is limited, the symptom can be easily selected and information on the appropriate remedy can be obtained. When the type of behavior is “Action”, the higher the score can be given to the action, the higher the predicted effect of the remedy is.

For example, in the data ID=1, the behavior ID=16 and the score=0.8. This means that the score indicating the degree of priority of the behavior (the symptom) of “Feel nausea” (the behavior ID=16) when the state is the first state “Using the drug name B (smoking-cessation aid 1)” (the state ID=3) (the score=1), the state is the second state “Smoking” (the state ID=1) (the score=1), and the state is the third state “Under a high stress” (the state ID=16) (the score=1) is 0.8. Different from the data ID=1, the data ID=2 indicates that the score for the behavior of “Feel nausea” when the state is not “Smoking” (the score=0) is 0.4. That is, this means that the degree of priority of the behavior (the symptom) of “Feel nausea” is lower in the state associated with the data ID=2 than that of the data ID=1.

Furthermore, in the data ID=9, the behavior ID=17 and the score=0.6. This means that the score indicating the level of the degree of priority of the behavior of “Having a headache” when the state is the first state “Using the drug name B (smoking-cessation aid 1)” (the state ID=3) and the state is the second state “Smoking” (the state ID=1) (the score=1) is 0.6. Accordingly, this means that the degree of priority of the behavior of “Feel nausea” (the behavior ID=16) when the state is “Using the drug name B (smoking-cessation aid 1)” (the state ID=3) (the score ID=1), the state is the second state is “Smoking” (the state ID=1) (the score=1), and the state is the third state “Under a high stress” (the state ID=16) (the score=1) is higher than the degree of priority of the behavior of “Have a headache” (the behavior ID=17).

The same applies when the type of behavior is “Action”. For example, as the behavior (the action) when nausea is felt (the state ID=12, the score=1), the drug name A (antiemetic) is kept (the state ID=7, the score=1), and the drug name A (the antiemetic) is being taken (the state ID=4, the score=1), the score for “Guided to take the drug name A (antiemetic)” (the table ID=14, the behavior ID=14) is 0.0 and the score for “Guided to stop taking the drug” (the table ID=17, the behavior ID=1) is 0.7. Accordingly, this means that, in the state where the user feels nausea but has already taken the drug name A (antiemetic), it is predicted that the action “Guided to stop taking the drug” is more effective than the action “Guided to take the drug name A (antiemetic)”. Additionally, in a case where the user has a renal dysfunction (the state ID=17), even when the user feels nausea, there is a possibility that dosing the drug name A (the antiemetic) should be avoided. Accordingly, as indicated in the data ID=26, the score for “Guided to consult with a doctor” (the behavior ID=18) is set high to urge the consultation to the doctor. Meanwhile, as indicated in the data ID=27, when the user does not have a renal dysfunction, the score for “Guided to consult with a doctor” (the behavior ID=18) is set low such that the action “Guided to take the drug name A (antiemetic)” is preferentially selected.

While the present embodiment uses the first to the third states as the states, four or more states may be included or the states may be limited to only two or less.

In the present embodiment, for example, the doctor preliminarily prepares the behavior-to-state score table such that further high scores are assigned to the behavior as the symptom believed to have the high degree of priority and the remedy believed to provide the high effect in the first to the third states. However, as long as the behavior-to-state score table can be generated, the behavior-to-state score table may be generated by any method, such as use of a model generated through machine learning. Additionally, the score for the behavior as the symptom may be changed based on the selection of the symptom by the user or the score for the behavior as the action may be changed based on the confirmed effect.

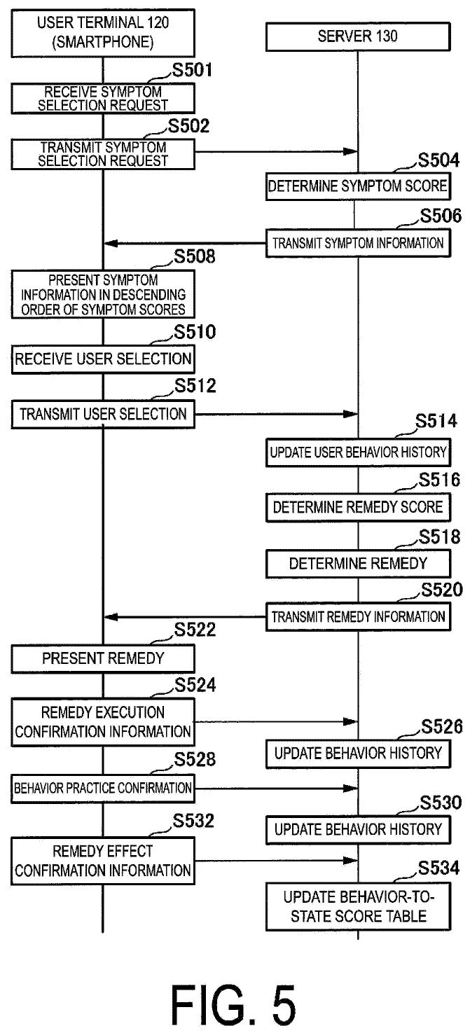

Next, operations of a system according to the present embodiment will be described with reference to FIG. 5. In the present embodiment, the operations by one user terminal 120 will be described for explanation, but the two or more user terminals 120 may be included.

Here, the user as the patient is Taro Yamada who is a smoking-cessation patient, during an outpatient treatment by a healthcare provider, the attribute data and the past medical history data were registered as shown in Tables 1 and 2 with the user ID=1. Furthermore, during the outpatient treatment, the drug name A (the antiemetic) and the drug name B (the smoking-cessation supplement 1) were prescribed by 12 tablets and 24 tablets, respectively, and registered as the prescription drug data (Table 3). The user downloads and installs an application required to embody the present embodiment to own user terminal 120. At this time, own user ID is input via the application to cause the storage unit 325 in the user terminal to store the user ID. After that, when the user measures an exhaled air CO concentration with an exhaled air CO concentration measuring apparatus at a predetermined time every day, information indicating the measured value is transmitted to the server 130 together with the user ID, and is registered with the biometric information data (Table 5) for the user.

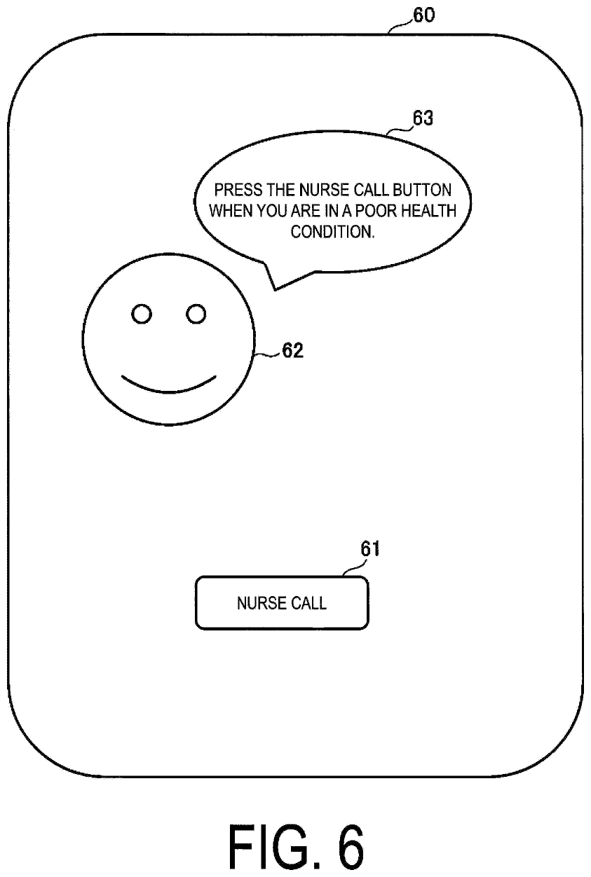

When the user is in a poor health condition due to, for example, a side effect of a drug in association with the treatment, the user inputs a symptom selection request and the input unit 321 in the user terminal 120 receives it (S501). The symptom selection request is an input to indicate that the user desires to select the symptom developed in association with the treatment to obtain information for the remedy. FIG. 6 illustrates an example of an application screen. When the application starts, the display unit 322 displays a nurse call button 61, a character 62, and a balloon 63 representing a remark of the character. The user can obtain the information on the remedy for the symptom that has occurred himself/herself through conversations with the character 62. When the user is in a poor health condition, by touching the nurse call button 61, the user terminal 120 receives the symptom selection request via the input unit 321.

When the user terminal 120 receives the symptom selection request, the user terminal 120 transmits it together with the user ID to the server 130 via the communication unit 323 (S502). When the server 130 receives the symptom selection request via the communication unit 333, the server 130 identifies the user based on the user ID included in it, and the symptom score determination unit 335 determines a symptom score for the user (S504). The symptom score determination is determined based on user history information for the user. In the present embodiment, first, the symptom score determination unit 335 in the server 130 generates a user state table as state information indicating the current state of the user based on the user history information, and stores it in the storage unit 337. Table 10 shows an example of the user state table.

TABLE 10
User State Table

| State | | |
|---|---|---|
| ID | State | Active |
| 1 | Smoking | 1 |
| 2 | Taking the drug name D (smoking-cessation aid | 0 |
| | 2) | |
| 3 | Using the drug name B (smoking-cessation aid | 1 |
| | 1) | |
| 4 | Taking the drug name A (antiemetic) | 0 |
| 5 | Keeping the drug name D (smoking-cessation | 0 |
| | aid 2) | |
| 6 | Keeping the drug name B (smoking-cessation | 1 |
| | aid 1) | |
| 7 | Keeping the drug name A (antiemetic) | 1 |
| 8 | Lack of sleep | 0 |
| 9 | Swollen | 0 |
| 10 | Having nightmares | 0 |
| 11 | Want to smoke | 1 |
| 12 | Feeling nausea | 0 |
| 13 | Having a headache | 0 |
| 14 | Affected with Interstitial pneumonia | 1 |
| 15 | Suffer from chronic constipation | 0 |
| 16 | Under a high stress | 1 |
| 17 | Have a disorder in the kidney function | 0 |
| 18 | Addicted to nicotine | 1 |
| 19 | Feeling irritated | 0 |
| 20 | Taking the drug name C (antidepressant) | 0 |
| 21 | Keeping the drug name C (antidepressant) | 0 |
| . . . | . . . | . . . |

The user state table is generated by being associated with each user and includes the state ID, the state, and the activation. The activation indicates whether the state is active, that is, whether the user is in the state. 1 means that the user is in the state (active) and 0 means that the user is not in the state (inactive). For example, the user state table shown in Table 10 is for the user with the user ID=1. Since the active information for the user with the state ID=1 and the state=“Smoking” is 1, this indicates that the user is in the state of smoking.

The user state table is generated based on the user history information with reference to the history-to-state table (Table 8). In the history-to-state table, in a case where the disease ID is assigned to the state ID, the past medical history data (Table 2) is referred to, in a case where the drug ID is assigned, the prescription drug data (Table 3) is referred to, and in a case where the behavior ID is assigned, the behavior history data (Table 6) is referred to. Then, the user history information is referred to for the history ID assigned to the state ID, and whether the user is in the state identified by each state ID is determined. When the duration indicated in a state table is over, the state is eliminated and therefore is not regarded as active.

For example, regarding the state ID=1, with reference to the state ID=1 in the history-to-state table, since the behavior ID=5 is assigned as the history ID, whether the user practices the behavior ID=5 is determined with reference to the behavior history table. As shown in Table 6, it is seen that the user practices the behavior “Smoke cigarettes” at two timings, which are timestamp=“12/12/2018 12:12:01” and “12/14/2018 12:12:00”. Then, with reference to the state list table, the duration of 72 hours is defined for the state ID=1. Thus, whether 72 hours have elapsed after “12/14/2018 12:12:00”, which is the last timestamp for “Smoke cigarettes”, is determined. When 72 hours have not passed, it is determined that the state is “Smoke cigarettes” and determined as active=1, and when 72 hours or more have passed, it is determined as 0.

When the drug ID is assigned in the history-to-state table, the state can be determined based on the amount of drug kept by the user in addition to the duration. For example, the drug name A (the antiemetic) was prescribed to the user with the user ID=1 identified by the data ID=1 in the prescription drug data by 12 tablets at Oct. 1, 2018. With reference to the behavior history of the user, the number of times that the drug name A (the antiemetic) was taken is counted, and the estimated remaining amount of the drug name A (the antiemetic) is calculated. When the estimated remaining amount is 0, the state of the state ID=7 “Keeping the drug name A (antiemetic)” is not determined to be active.

While the user attribute data is not used in the present embodiment, it may be used or other information can be used. For example, a period during which the state can be continued may be changed based on an age and a weight of the user. Furthermore, the user terminal may store the user history information, the user terminal may generate the user state table, and the user state table may be transmitted to the server together with the symptom selection request.

Next, the behavior table and the behavior-to-state score table are referred to based on the user state table to generate a symptom score table for the user. First, with reference to the behavior table, the behavior with the type of “Symptom” is extracted. In Table 4, the behavior IDs=11 to 17 and 19 are the symptoms, and therefore are selected. Then, with reference to the behavior-to-state score table for these behavior IDs, the scores of the respective behaviors as the symptoms are extracted. At that time, the user state table is referred to based on the state ID associated with each symptom to obtain the current state of the user, and the score for the symptom corresponding to the user state is determined among the scores for the respective symptoms to generate the symptom score table.

For example, to determine the score for the symptom of the behavior ID=16 and the behavior “Feel nausea”, the data IDs=1 to 8 with the behavior ID=16 are referred to in the behavior-to-state score table. Since the state associated with the behavior ID=16 is the state ID=1 (Smoking), the state ID=3 (Using the drug name B (smoking-cessation aid 1), and the state ID=16 (Under a high stress), the states are obtained with reference to the user state table. With reference to Table 10, since the state of the user is 1 (active) in the state ID=1 (Smoking), 1 (active) in the state ID=3 (Using the drug name B (smoking-cessation aid 1), and 1 (active) in the state ID=16 (Under a high stress), the score=0.8 of the data ID corresponding to them is determined as the score for the behavior ID=16. The scores are similarly determined for other symptoms, and the symptom score table including the scores for the respective symptoms of the user is generated. Table 11 shows an example of the symptom score table.

TABLE 11
Symptom Score Table

| Table | Behavior | | |
|---|---|---|---|
| ID | ID | Behavior | Score |
| 1 | 11 | Sleepy | 0.1 |
| 2 | 12 | Got swollen | 0.4 |
| 3 | 13 | Have nightmares | 0.1 |
| 4 | 14 | Cannot Sleep | 0.3 |
| 5 | 15 | Want to smoke | 0.7 |
| 6 | 16 | Feel nausea | 0.8 |
| 7 | 17 | Have a headache | 0.6 |
| 8 | 19 | Feel irritated | 0.2 |

The symptom score table includes the behavior ID of the behavior as the symptom, the behavior, and the score. For example, the Table ID=1 indicates that the score for the symptom “Sleepy” as the behavior ID=11 is 0.1.

When the server 130 generates the symptom score table, the server 130 transmits it to the user terminal 120 as symptom information (S506). When the user terminal 120 receives the symptom information, the user terminal 120 preferentially presents the behaviors as the symptoms in the display unit in descending order of the scores shown in a score table (S508). The user selects the symptom that is thought to correspond to the symptom currently developed himself/herself among the presented symptoms. The presentation to the user can also be presented by a voice.

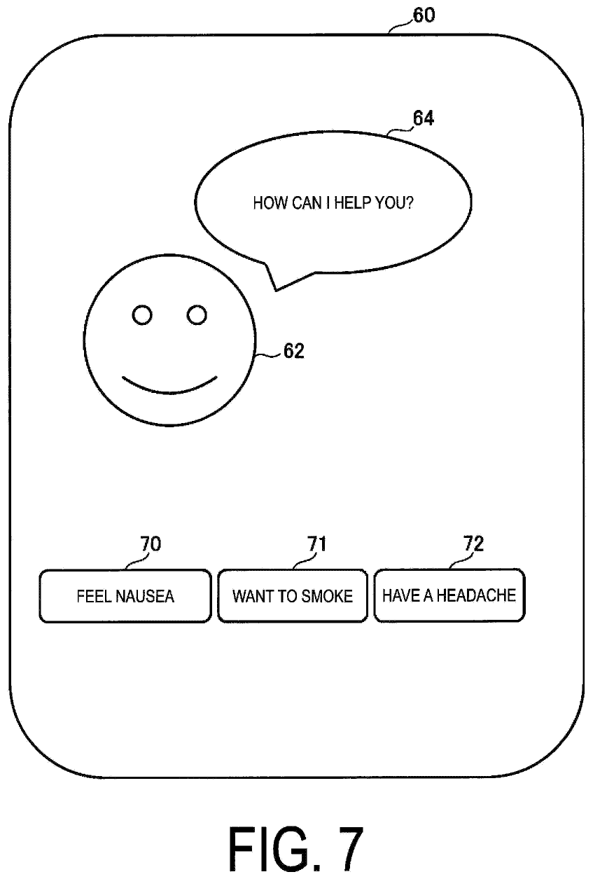

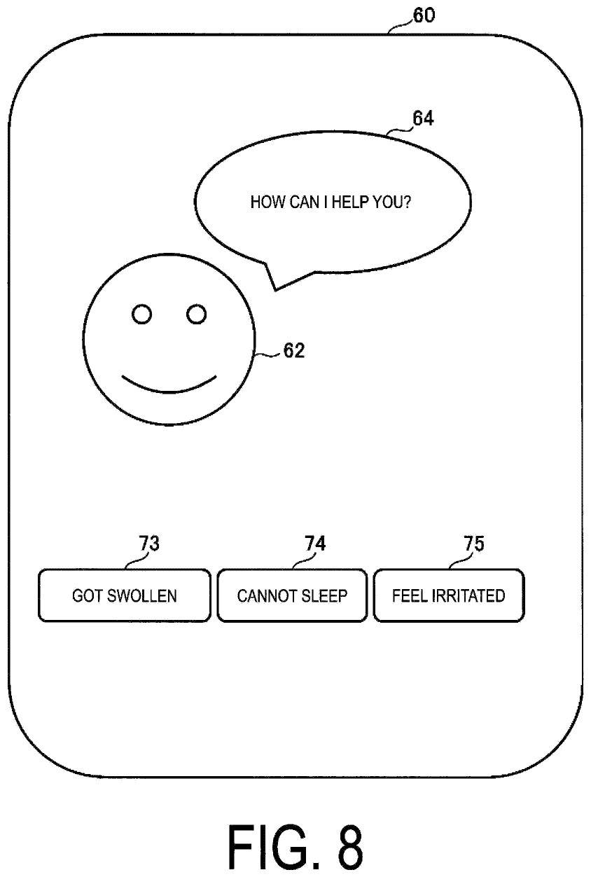

FIGS. 7 and 8 illustrate an example of a screen of the present embodiment that displays symptoms candidates for the symptom possibly developed in the user. Among the symptoms to be the candidates, the symptoms by the number that can be displayed in the display, the three symptoms here, are displayed in the display in descending order of the scores. For example, buttons (70 to 72) corresponding to the three symptoms with the highest symptom scores, “Feel nausea” (the score=0.8), “Want to smoke” (the score=0.7), and “Have a headache” (the score=0.6) are displayed. When the symptom developed in the user is not any of these symptoms, the user operates the user terminal 120 to display the next candidate symptoms. In the present embodiment, the user terminal 120 is the smartphone. Accordingly, performing a swipe operation on the touch panel allows displaying buttons (73 to 75) for the next candidate symptoms. The symptoms are displayed as the candidate symptoms in sequence in descending order of the scores. When the button corresponding to the symptom developed himself/herself is found, by touching the button, the symptom can be selected.

Since a size of the screen to present the information to the user is limited in an information terminal, all of the candidate symptoms cannot be presented at a time. Thus, while only some of the candidate symptoms are displayed, the displayed candidate symptoms need to be changed by, for example, a scroll operation by the user. However, it is not always easy to search for and select the symptom currently developed himself/herself among a large number of the candidate symptoms when the symptom in association with the treatment is developed and the user is in a poor health condition. In particular, when the user is a patient who suffers from a plurality of diseases or a patient who takes a plurality of drugs, an extremely large number of symptoms become the candidates. In a case where the developed symptom is numbness of the hand or dizziness, selecting the symptom while performing the scroll operation is even more difficult.

The use of the present embodiment preferentially presents the symptom that is more likely to be currently developed in the user, the symptom that limits the ability of the user to select the symptom, and the symptom having a high risk of hazardous to life. Therefore, even when the size of the screen is limited, a necessity of, for example, the scroll operation can be reduced, allowing the user to select the symptom developed himself/herself more easily.

When the user terminal includes a sufficiently large display, all candidate symptoms may be displayed at a time. In that case, for example, the display can be performed in descending order of the scores from the top. In this case as well, the user refers to the symptoms in order from the top, thereby ensuring quickly selecting the symptom developed himself/herself.

When the user terminal 120 receives the selection of the symptom by the user (S510), the user terminal 120 transmits it to the server 130 (S512). When the server 130 receives the user selection, the server 130 updates the behavior history of the user based on it (S514). For example, when the symptom selected by the user is “Feeling nausea”, new data ID is added to the behavior history data (Table 6), and the user ID=1, the behavior ID=16, the behavior=“Feel nausea”, and the timestamp=the current time are associated with the new data ID and registered.

Further, based on the user selection, the score for the symptom in the behavior-to-state score table may be updated. For example, the score for the symptom selected by the user may be increased and scores for the unselected symptoms may be decreased. This allows automatically adjusting the scores for the symptoms and assigning more realistic values.

Then, the remedy determination unit 336 in the server 130 determines a remedy score to determine the remedy to alleviate the symptom currently developed in the user (S516). In the present embodiment, first, the user state table is updated based on the updated behavior history data. The user state table can be updated by the process similar to the generation of the user state table described above. Based on the updated user state table, the score for each behavior as the action is determined with reference to the behavior table and the behavior-to-state score table. That is, the behavior ID of the behavior whose type is “Action” is extracted with reference to the behavior table. Among the data IDs associated with the behavior IDs of the behaviors as the actions in the behavior-to-state score table, the data ID corresponding to the current state of the user is selected based on the updated user state table and the score for the behavior is selected.

For example, based on the updated behavior history, the state of the state ID=12 (Feeling nausea) in the user state table is updated to 1 (active). With reference to the behavior table (Table 4), the behavior IDs=1 to 4 and 18 whose type=“Action” are extracted, and the data IDs 13 to 20 corresponding to them are extracted in the behavior-to-state score table. With reference to the user state table, the behavior score=0.8 for the behavior ID=4 (Guided to take the drug name A (antiemetic)) in the data ID=13 and the behavior ID=0.1 for the data ID=18 (Guided to stop taking the drug) corresponding to the state ID=12 (Feeling nausea) and the state=1 (active), the state ID=7 (Keeping the drug name A (antiemetic)) and the state=1, and the state ID=4 (Taking the drug name A (antiemetic)) and the state=0 are extracted.

Table 12 shows an example of a remedy score table indicating the scores for the respective behaviors. The remedy score table includes the behavior ID, the behavior, and the score.

TABLE 12
Remedy Score Table

| Table | Behavior | | |
|---|---|---|---|
| ID | ID | Behavior | Score |
| 1 | 1 | Guided to stop taking the drug | 0.1 |
| 2 | 2 | Guided to drink water | 0.7 |
| 3 | 3 | Guided to cool down | 0.2 |
| 4 | 4 | Guided to take the drug name A | 0.8 |
| | | (antiemetic) | |

The server 130 refers to the remedy score table, selects the behavior ID corresponding to the table ID with the highest score, and transmits remedy information stored in the storage unit 337 corresponding to the selected behavior ID to the user terminal 120 (S520). When the user terminal 120 receives this, the user terminal 120 presents information showing the remedy to the user.

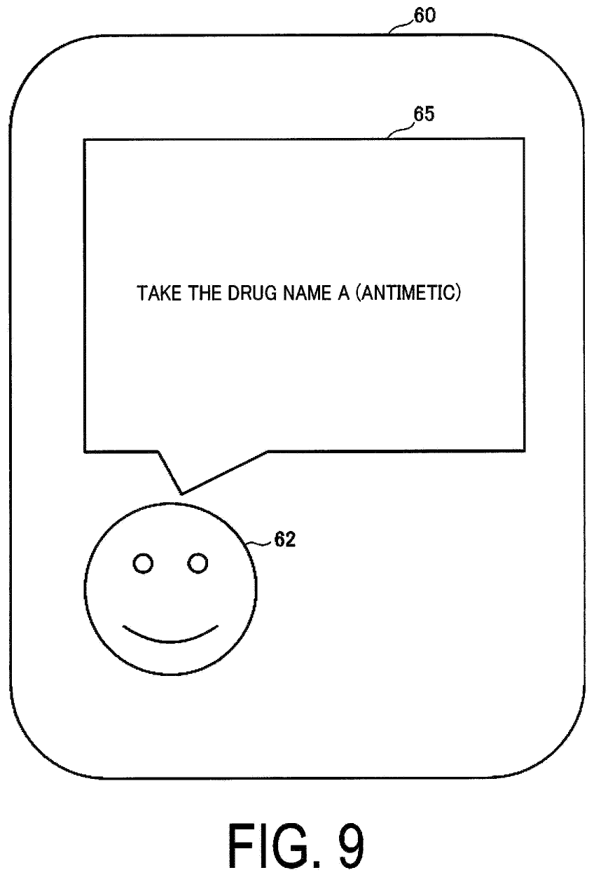

FIG. 9 illustrates an example of a screen of the user terminal to present the information on the remedy presented to the user. The server 130 determines that the user is caused to take the remedy corresponding to the behavior ID=4 with the highest score “Guided to take the drug name A (antiemetic)” based on the remedy score table shown in Table 12. The server 130 obtains the remedy information associated with the remedy. The remedy information includes information to show the remedy to the user. Here, the server 130 obtains “Dose the drug name A (antiemetic)” as the remedy information corresponding to the determined remedy, and transmits it to the user terminal 120. The user terminal 120 receives it, and as illustrated in FIG. 9, causes the display to display it (S522) to allow the user to practice the behavior “Guided to take the drug name A (antiemetic)”. The presentation to the user may be presented by a voice.

When the user terminal 120 presents the remedy information to the user, the user terminal 120 transmits remedy taking confirmation information to the server 130 (S524). When the server 130 receives it, the server 130 updates the behavior history data of the user to show that the remedy was taken (S526).

Further, after an elapse of a predetermined time after the presentation of the information showing the action to the user, the user terminal 120 prompts the user to confirm whether the behavior guided in the remedy was practiced. When the user does not practice the behavior, the user terminal 120 presents information prompting the user to practice the behavior. When the user terminal 120 receives an input indicating the completion of practice, the user terminal 120 transmits behavior practice confirmation information to the server 130 (S528). When the server 130 receives it, the server 130 updates the behavior history data to show the behavior practiced (S530). Table 13 shows an example of the updated behavior history data.

TABLE 13
Behavior History Data

| Data | User | Behavior | | |
|---|---|---|---|---|
| ID | ID | ID | Behavior | Timestamp |
| 1 | 1 | 5 | Smoke cigarettes | 12/12/2018 12:12:01 |
| 2 | 1 | 9 | Apply the drug name B | 12/12/2018 23:12:01 |
| | | | (smoking-cessation aid 1) | |
| 3 | 1 | 9 | Apply the drug name B | 12/13/2018 11:12:01 |
| | | | (smoking-cessation aid 1) | |
| 4 | 1 | 5 | Smoke cigarettes | 12/14/2018 12:12:00 |
| 5 | 1 | 9 | Apply the drug name B | 12/14/2018 23:12:01 |
| | | | (smoking-cessation aid 1) | |
| 6 | 1 | 16 | Feel nausea | 12/14/2018 23:26:01 |
| 7 | 1 | 4 | Guided to take the drug | 12/14/2018 23:34 |
| | | | name A (antiemetic) | |
| 8 | 1 | 10 | Dose the drug name A | 12/14/2018 23:39 |
| | | | (antiemetic) | |

In a behavior practice confirmation (S528), in a case where it is confirmed that the user has practiced the behavior guided as the remedy, the user terminal 120 further prompts the user to input to confirm whether the symptom has been alleviated by the behavior after an elapse of a predetermined time. The predetermined time can be determined based on the behavior practiced. For example, in a case where the effect appears soon, the predetermined time is set to after five minutes, and when the effect does not appear after an elapse of 12 hours, the predetermined time is set to 13 hours. When the user terminal 120 receives the user input, the user terminal 120 transmits effect confirmation information to the server 130 (S532). The server 130 can update the behavior-to-state score table based on the received effect confirmation information (S534). For example, the score for the effective remedy can be increased and the score for the ineffective remedy can be reduced in the behavior-to-state score table. The score can be defined for each user, and the score can be updated based on the effect only for each user.

The use of the present embodiment allows the user to easily select the symptom developed in association with the treatment, and allows taking the remedy believed to be effective to alleviate the selected symptom.

In the present embodiment, the storage unit in the server 130 stores the various tables, but the storage unit in the user terminal 120 may store some or all of these tables.

In the present embodiment, the symptoms developed in association with the treatments include the symptoms other than a side effect of a drug as the symptoms developed in association with the treatments, but the aspect can be limited to the side effect of the drug, and can be only the symptoms developed in association with the treatments not including side effects of the drug.

In the present embodiment, the table, such as a behavior-to-state table, is managed as a common table with the symptoms and the actions as the behaviors. However, generating tables for the respective symptoms and actions allows the tables to be executed by similar information processing.

As a modification, instead of the behavior-to-state score table, a behavior-to-behavior score table may be used to determine the symptom and the remedy. It can be achieved by simply managing the behavior history without management of the states of the user. This can be achieved by similar process except that the process associated with the state in the above-described embodiments is unnecessary and the behavior is associated with the behavior, not the state. Instead of the behavior-to-state score table, for example, the following behavior-to-behavior score table can be used.

TABLE 14
Behavior-to-Behavior Score Table

| Data | First behavior | | Second behavior | | Third behavior | | Fourth behavior | |
|---|---|---|---|---|---|---|---|---|
| ID | ID | Score | ID | Score | ID | Score | ID | Score |
| 1 | 16 Feel | 0.8 | 5 Smoke | 1 | 9 Apply | 1 | 10 Dose | 1 |
| | nausea | | cigarettes | | the drug name | | the drug name | |
| | | | | | B (smoking- | | A (antiemetic) | |
| | | | | | cessation aid 1) | | | |
| 2 | 16 | 0.4 | 5 | 1 | 9 | 0 | 10 | 1 |
| 3 | 16 | 0.4 | 5 | 0 | 9 | 1 | 10 | 1 |
| 4 | 16 | 0.3 | 5 | 0 | 9 | 0 | 10 | 1 |
| 5 | 16 | 0.7 | 5 | 1 | 9 | 1 | 10 | 0 |
| 6 | 16 | 0.3 | 5 | 1 | 9 | 0 | 10 | 0 |
| 7 | 16 | 0.3 | 5 | 0 | 9 | 1 | 10 | 0 |
| 8 | 16 | 0.2 | 5 | 0 | 10 | 0 | 10 | 0 |
| 9 | 17 Have | 0.6 | 5 | 1 | 9 | 1 | — | — |
| | a headache | | | | | | | |
| 10 | 17 | 0.4 | 5 | 1 | 9 | 0 | — | — |
| 11 | 17 | 0.4 | 5 | 0 | 9 | 1 | — | — |
| 12 | 17 | 0.3 | 5 | 0 | 9 | 0 | — | — |
| . . . | . . . | . . . | . . . | . . . | . . . | . . . | . . . | . . . |

In Table 14, the first behavior to the fourth behavior are made to correspond to the data ID and stored. When the behaviors identified by the respective IDs have been practiced by the user, the scores for the second to fourth behaviors are 1, and when not practiced, the scores are 0. In a case where the scores are indicated by the scores for the second to fourth behaviors, the score for the first behavior indicates the score for the first behavior ID. When the type of the behavior is a symptom, the score indicates the level of the degree of priority that the behavior is presented to the user, and when the type is the action, the score indicates the level of the expected effect.

To determine the symptom score table for each user, whether each behavior has been practiced can be determined based on the behavior history data. When a predetermined time or more has passed after the timestamp in the behavior history data, it can be determined that the behavior is not practiced. Other processes are similar to the above-described embodiments.

As another one modification, in the embodiments described above, the two computers, the user terminal 120 and the server 130, are used. However, one computer, for example, the user terminal 120 having all functions may perform all processes in the user terminal 120 without the use of the server 130. This allows omitting the information transmission/reception processes with the server 130. Other processes are similar to the above-described embodiments.

In the processes or operations described above, as long as an inconsistency in the process or the operation, such as use of data that cannot be used in a certain step yet, does not occur, the process or the operation can be freely changed. The respective embodiments described above have been described for illustrative purposes of the present invention, and the present invention is not limited to these embodiments. The present invention can be implemented in various forms without departing from the spirit thereof.

### REFERENCE SIGNS LIST

100 System
110 Network
120 User terminal
121 Processing device
122 Display device
123 Input device
124 Storage device
125 Communication device
128 Bus
130 Server
131 Processing device
132 Display device
133 Input device
134 Storage device
135 Communication device
138 Bus
321 Input unit
322 Display unit
323 Communication unit
324 Control unit
325 Storage unit
331 Input unit
332 Display unit
333 Communication unit
334 Control unit
335 Symptom score determination unit
336 Remedy determination unit
337 Storage unit

1. A system comprising:
a server; and
a user terminal, wherein
the system is for alleviating a symptom developed in a user in association with a treatment involving a side effect of a drug, and
the server determines symptom scores for two or more respective symptoms based on user history information including information on a drug, and the symptom scores indicate degrees of priority presented to the user, wherein
the user terminal:
presents information indicating the symptoms in descending order of the determined symptom scores to allow the user to select a symptom currently developed in the user; and
receives a user selection indicating the symptom selected by the user based on the information indicating the presented symptoms, wherein
the server determines remedy information based on the user selection and the user history information, the remedy information indicates a remedy to alleviate the selected symptom, and
the user terminal presents the remedy to alleviate the symptom based on the determined remedy information.

2. The system according to claim 1, wherein
the determining the symptom scores include determining the symptom scores for the two or more respective symptoms based on state information, and the state information indicates states of the user determined based on the user history information.

3. The system according to claim 2, wherein
the server stores a score table, and the score table indicates the symptom scores for the respective symptoms predetermined for the respective states of the user, and
the determining the symptom scores include selecting the symptom scores for the respective symptoms of the user from the score table based on the state information.

4. The system according to claim 3, wherein
the server changes the predetermined symptom score for each of the symptoms based on the user selection.

5. The system according to claim 1, wherein
the determining the remedy information includes determining remedy scores indicating levels of expected effects for two or more respective remedies based on state information indicating states of the user determined based on user history information, and selecting the remedy information indicating a remedy with a highest remedy score.

6. The system according to claim 5, wherein
the server stores a score table, and the score table indicates the remedy scores for the respective remedies for the respective symptoms predetermined for respective states of the user, and
the determining the remedy information includes selecting the remedy with the highest remedy score of the user from the score table based on the user selection and the state information.

7. The system according to claim 6, wherein
the server changes the predetermined remedy score for each of the remedies for each of the symptoms based on effect confirmation information, and the effect confirmation information indicates an effect of a remedy taken by the user.

8. The system according to claim 1, wherein
the determining the symptom scores include determining the symptom scores for the respective two or more symptoms based on presence or absence of behaviors of the user determined based on the user history information.

9. The system according to claim 1, wherein
the determining the remedy information includes determining remedy scores that indicates levels of predicted effects for the two or more respective remedies based on presence or absence of behaviors of the user determined based on the user history information, and selecting the remedy information indicating a remedy with a highest remedy score.

10. The system according to claim 1, wherein
the user history information includes at least one of attribute information, past medical history information, prescription information, biometric information, and behavior history information, and the behavior history information indicates behaviors practiced by the user.

11. The system according to claim 10, wherein
the biometric information includes an exhaled air carbon monoxide concentration of the user measured by a carbon monoxide concentration measuring apparatus.

12. The system according to claim 1, wherein
the remedy information is further determined based on prescription information indicating a type and an amount of a drug prescribed to the user.

13. A server for alleviating a symptom developed in a user in association with a treatment involving a side effect of a drug, wherein
the server:
determines symptom scores for two or more respective symptoms based on user history information including information on a drug, the symptom scores indicating degrees of priority presented to the user;
transmits the symptom scores to a user terminal to present information indicating the symptoms in descending order of the symptom scores;
receives a user selection indicating the symptom selected by the user based on the information indicating the presented symptoms from the user terminal;
determines remedy information based on the user selection and the user history information, the remedy information indicating a remedy to alleviate the selected symptom; and
transmits the remedy information to the user terminal.

14. A user terminal for alleviating a symptom developed in a user in association with a treatment involving a side effect of a drug, wherein
the user terminal:
receives symptom scores for two or more respective symptoms determined based on user history information including information on a drug from a server, the symptom scores indicating degrees of priority presented to the user;
presents information indicating the symptoms in descending order of the determined symptom scores to allow the user to select a symptom currently developed in the user;
receives a user selection indicating the symptom selected by the user based on the information indicating the presented symptoms;
transmits the user selection to the server to determine remedy information based on the user selection and the user history information, the remedy information indicating a remedy to alleviate the selected symptom;
receives the determined remedy information from the server; and
presents the remedy to alleviate the symptom based on the remedy information.

15. A method for alleviating a symptom developed in a user in association with a treatment involving a side effect of a drug, the method being executed by one or more computers, the method comprising:
determining symptom scores for two or more respective symptoms based on user history information including information on a drug, the symptom scores indicating degrees of priority presented to the user;
presenting information indicating side effects in descending order of the determined symptom scores to allow the user to select a symptom currently developed in the user;
receiving a user selection indicating the side effect selected by the user based on the information indicating the presented symptoms;
determining remedy information based on the user selection and the user history information, the remedy information indicating a remedy to alleviate the selected symptom; and
presenting the remedy to alleviate the symptom based on the determined remedy information.

16. A method for alleviating a symptom developed in a user in association with a treatment involving a side effect of a drug, the method being executed by a server, the method comprising:
determining symptom scores for two or more respective symptoms based on user history information including information on a drug, the symptom scores indicating degrees of priority presented to the user;
transmitting the symptom scores to a user terminal to present information indicating the symptoms in descending order of the symptom scores;
receiving a user selection indicating the symptom selected by the user based on the information indicating the presented symptoms from the user terminal;
determining remedy information based on the user selection and the user history information, the remedy information indicating a remedy to alleviate the selected symptom; and
transmitting the remedy information to the user terminal.

17. A method for alleviating a symptom developed in a user in association with a treatment involving a side effect of a drug, the method being executed by a user terminal, the method comprising:
receiving symptom scores for two or more respective symptoms determined based on user history information including information on a drug from a server, the symptom scores indicating degrees of priority presented to the user;
presenting information indicating the symptoms in descending order of the determined symptom scores to allow the user to select a symptom currently developed in the user;
receiving a user selection indicating the symptom selected by the user based on the information indicating the presented symptoms;
transmitting the user selection to the server to determine remedy information based on the user selection and the user history information, the remedy information indicating a remedy to alleviate the selected symptom;
receiving the determined remedy information from the server; and
presenting the remedy to alleviate the symptom based on the remedy information.

18. A non-transitory computer-readable medium storing a set of programs that cause one or more processors to execute the method according to claim 15.
